(19) United States
(12) Patent Application Publication
(10) Pub. No.: US 2023/0181812 A1
(43) Pub. Date: Jun. 15, 2023

(54) SURGICAL DRAPE ACCESSORY

(71) Applicant: Ray King, New York City, NY (US)

(72) Inventor: Ray King, New York City, NY (US)

(21) Appl. No.: 17/921,374

(22) PCT Filed: Apr. 26, 2021
(86) PCT No.: PCT/US21/29065

§ 371 Date: Oct. 26, 2022

Related U.S. Application Data
(60) Provisional application No. 63/015,632, filed on Apr. 26, 2020.

(51) Int. Cl. A61M 1/00; A61F 13/02; A61F 13/00; A61L 15/24

(52) U.S. Cl. CPC A61M 1/86; A61F 13/0206; A61F 13/00068; A61L 15/24

(57) ABSTRACT

The present invention relates generally to methods and systems for performance of collecting fluid emanating from or adjacent to a surgical site or wound. It does so utilizing a highly absorptive biocompatible sponge, such as polyvinyl alcohol or other material, which is connected to wall suction via a common hole running through the base of this sponge. The sponge configuration can vary (e.g. circular, linear, or other) and may be affixed to the drape via Velcro, magnetic, tape, staples or other method, so that abuts the drape surface; a raised portion may also serve as a “dam” to prevent fluid going to unwanted regions (e.g. onto the surgeon's feet, floor, or other undesired draped or undraped region).

### CROSS-REFERENCE TO RELATED APPLICATIONS

This application claims the benefit and priority of U.S. provisional patent application Ser. No. 63/015,632, filed Apr. 26, 2020, entitled “SURGICAL DRAPE ACCESSORY”, the contents of which is hereby incorporated by reference in its entirety.

### FIELD OF THE INVENTION

This invention relates generally to methods and systems for suctioning fluid adjacent to a surgical incision or site, more particularly to a drape accessory with a sponge affixed to the surgical drape as desired, and containing fluid to prevent dripping on the floor, onto a surgical provider, or on another unwanted region.

### BACKGROUND OF THE INVENTION

Irrigation, blood, or other fluids emanating from or around a surgical incision or other wound often drip into unwanted areas (e.g. surgeon's foot, different region of the body or drape, and/or floor) during surgical procedures. While surgeons actively suction fluid from a surgical incision or wound, often times liquid can escape off the side.

### SUMMARY OF THE INVENTION

The present invention relates to methods and systems for utilizing a drape accessory, effectively creating a barrier in key locations around a surgical site, which is under continuous suction so that fluid cannot escape.

In one aspect of the invention, the sponge material may be made using a substance such as a natural sponge or synthetic sponge, such as those made from polyvinyl alcohol (which is biocompatible and capable of absorbing greater fluid than normal natural sponges), connected to continuous wall suction, via a communicating orifice (tunnel) inside the sponge running longitudinally, with either a dead end on the other side or circumferentially connected to the same orifice (e.g. in an oval, circle or other loop around the incision).

In some exemplary embodiments, the sponge may be placed in configurations as desired, including oval, linear, etc. affixed via hook and loop fasteners (e.g. Velcro), tape, magnets, staples, or other mechanism and adjustable by surgeon.

In some exemplary embodiments, the perforations or holes may be connected via the suction tubing to a “Y” connector. Typically the suction tubing would not enter deep into the tunnel, but barely so to provide suction.

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

FIG. 1 illustrates a cross-sectional view of a drape accessory of the present invention;

FIG. 2 illustrates linear drape accessories connected to a common suction source; and

FIG. 3 illustrates a looped drape accessory connected to a suction source.

### DETAILED DESCRIPTION OF THE INVENTION

The detailed description set forth below is intended as a description of the presently exemplified systems, devices and methods provided in accordance with aspects of the present invention and are not intended to represent the only forms in which the present invention may be prepared or utilized. It is to be understood, rather, that the same or equivalent functions and components may be accomplished by different embodiments that are also intended to be encompassed within the spirit and scope of the invention. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplary methods, devices and materials are now described. All publications mentioned herein are incorporated herein by reference for the purpose of describing and disclosing, for example, the designs and methodologies that are described in the publications which might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

The present invention relates to methods and systems for utilizing a drape accessory, effectively creating a barrier in key locations around a surgical incision or other surgical site, which is under continuous suction so that fluid cannot escape. In a typical surgical setting, an incision is marked and draped around the incision; several suction devices (typically to wall suction, with an intervening suction cannister) are present; while surgeons may utilize hand-held suction devices.

In one aspect of the invention, the sponge material of the drape accessory may be made using a substance such as a natural sponge or synthetic sponge, such as those made from polyvinyl alcohol (which is biocompatible and capable of absorbing greater fluid than normal natural sponges), connected to continuous wall suction, via a communicating orifice (e.g. channel or tunnel) inside the sponge running longitudinally, with either a dead end on the other side or circumferentially connected to the same orifice (e.g. in an oval, circle or other loop around the incision).

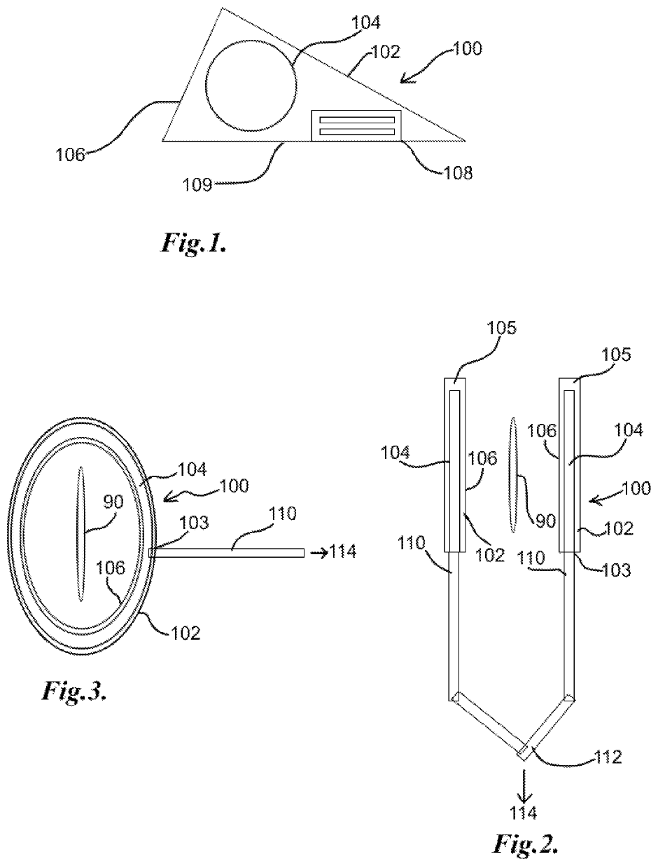

FIG. 1 illustrates a cross-sectional view of a drape accessory 100 having a sponge material barrier 102, and a communicating orifice shown as an internal channel 104. The sponge material barrier 102 may generally be porous and provide for fluid communication from the outside (e.g. open-cell pore structure), such as at the side facing the surgical field 106, into the internal channel 104 such that fluid absorbed by the sponge material barrier 102 may be pulled into the internal channel 104. The internal channel 104 may run substantially the length of the drape accessory 100, as illustrated in the schematic view of FIG. 2, and may connect to a suction source 110 which may be partially inserted into the internal channel 104. In some embodiments, the drape accessory 100 may be substantially linear, as illustrated in FIG. 2, and may include a stop or dead end 105 to the internal channel 104 such that fluid may not exit from the closed end and/or to increase the suction within the internal channel 104. In other embodiments, the drape accessory 100 may form a loop, as illustrated in FIG. 3, and a suction source 110 may penetrate the sponge material barrier 102, such as at access point 103, to connect the suction source 110 to the internal channel 104.

In some exemplary embodiments, the sponge may be placed in configurations as desired, including oval, linear, etc. affixed via hook and loop fasteners (e.g. Velcro), tape, magnets, or other mechanism and adjustable by surgeon. FIG. 1 illustrates an example of the placement of an attachment mechanism 108. As shown, the attachment mechanism 108 may, for example, be sunken in to the sponge material barrier 102 such that the attachment mechanism 108 is substantially flush with the bottom surface 109.

In some exemplary embodiments, the perforations or holes may be connected via the suction tubing to a “Y” connector to a single suction source, as illustrated in FIG. 2 with suction sources 110, Y-connector 112 and suction connection 114. Typically the suction tubing would not enter deep into the tunnel, but barely so to provide suction, as illustrated in FIGS. 2 and 3 with suction source 110 and internal channel 104. This allows continuous passive suction while the operation is proceeding and does not require the manual operation of suction devices by the surgical personnel. The sponge material barrier 102 further creates a contained area with its sloped and/or vertical side surfaces of a height to form a raised portion such that fluid may generally not flow over before it is suctioned into the sponge material, and may thus act as a “dam” or other barrier to contain the fluid. The height may be selected for different applications or different expected levels of fluid, such as those exiting from a surgical site.

### EXAMPLE

A surgeon may choose, for example, to surround the abdominal incision of a ventriculoperitoneal (VP) shunt with a circumferential drape with the features of this invention; s/he would affix the sponge of the drape accessory 100 (e.g. using Velcro, magnet, tape, or other method for the attachment mechanism 108) to the drape totally surrounding the incision 90, and connect a suction tube 110 to the orifice that is open 103 (connected to a contiguous tunnel (internal channel 104) which runs the substantial or total length of the drape accessory 100 to connect to itself, with an adjacent outlet to the suction; fluid emanating from or adjacent to the incision 90 would then be suctioned into this mechanism, to wall suction, into a cannister, as illustrated in FIG. 3.

An alternative may be, for example, for a long (e.g. scoliosis) corrective spine operation, two long strips of sponge in the drape accessories 100 may be utilized and connected via a “Y” connector 112 to a common suction source 114, with comparable method as in prior paragraph used to fixate it to the drape, as illustrated in FIG. 2.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein. Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A system for draining fluid from around a surgical site comprising:
a sponge material barrier having a height and an outside surface;
an internal channel running substantially the length of said sponge material barrier, said internal channel being in fluid communication through said sponge material barrier to said outside surface; and
an access point for said internal channel for connection to a suction source.

2. The system of claim 1, further comprising suction tubing connected to said access point.

3. The system of claim 1, wherein said sponge material barrier is substantially linear and further comprises a dead end stop at one end of said internal channel and said access point at the other end.

4. The system of claim 1, wherein said sponge material barrier is in the form of a loop.

5. The system of claim 1, further comprising an attachment mechanism disposed on a bottom surface of said sponge material barrier.

6. The system of claim 5, wherein said attachment mechanism is sunk in and substantially flush with said bottom surface.

7. The system of claim 5, wherein said attachment mechanism is selected from the group consisting of hook and loop fastener, adhesive, magnet, tape and staples.

8. The system of claim 3, further comprising at least one additional barrier which is substantially identical to said sponge material barrier and connected to said suction source.

9. The system of claim 8, further comprising a Y-connector attached to suction tubing connected to said access points of said sponge material barrier and said at least one additional barrier.

10. The system of claim 1, wherein said sponge material barrier is constructed from a biocompatible natural or synthetic sponge material.

11. The system of claim 1, wherein said sponge material barrier is constructed from polyvinyl alcohol.

12. A method of collecting fluid emanating from a surgical site comprising:
placing a sponge material barrier having a height and an outside surface adjacent to a surgical site, said sponge material barrier comprising:
an internal channel running substantially the length of said sponge material barrier, said internal channel being in fluid communication through said sponge material barrier to said outside surface; and
an access point for said internal channel for connection to a suction source; and
applying suction via said access point to draw fluid emanating from said surgical site.

13. The method of claim 12, wherein said sponge material barrier is the form of a loop and is placed to substantially surround said surgical site.

14. The method of claim 12, wherein said sponge material barrier is substantially linear and further comprises a dead end stop at one end of said internal channel and said access point at the other end.

15. The method of claim 14, further comprising at least one additional barrier which is substantially identical to said sponge material barrier adjacent to and on a different side of said surgical site than said sponge material barrier.

16. The method of claim 15, further comprising utilizing a Y-connector attached to suction tubing connected to said access points of said sponge material barrier and said at least one additional barrier.

17. The method of claim 12, further comprising attaching said sponge material barrier to an area adjacent to said surgical site with an attachment mechanism selected from the group consisting of hook and loop fastener, adhesive, magnet, tape and staples.

18. The method of claim 12, wherein said sponge material barrier is constructed from a biocompatible natural or synthetic sponge material.

19. The method of claim 12, wherein said sponge material barrier is constructed from polyvinyl alcohol.

20. The method of claim 12, wherein said height is selected to retain said fluid emanating from said surgical site.
